(19) United States
(12) Patent Application Publication
(10) Pub. No.: US 2023/0190615 A1
(43) Pub. Date: Jun. 22, 2023

(54) COMPOSITIONS COMPRISING RHAMNOLIPID, ALKYL POLYGLYCOSIDE AND ACYL LACTYLATE

(71) Applicant: Evonik Operations GmbH, Essen (DE)

(72) Inventors: Stefan Julian Liebig, Duesseldorf (DE); Hans Henning Wenk, Muelheim an der Ruhr (DE); Kathrin Daniela Brandt, Duesseldorf (DE); Manuela Salmina-Petersen, Hamburg (DE); Jan Jänichen, Hamburg (DE); Ilona DAVIDS, Kerken (DE)

(73) Assignee: Evonik Operations GmbH, Essen (DE)

(21) Appl. No.: 17/907,015

(22) PCT Filed: Mar. 16, 2021
(86) PCT No.: PCT/EP2021/056635

§ 371 Date: Sep. 22, 2022

(30) Foreign Application Priority Data
Mar. 24, 2020 (EP) 20165164.3

(51) Int. Cl. A61K 8/60; A61K 8/37; A61Q 17/04; A61Q 19/08; A61Q 15/00; A61Q 19/00; A61Q 17/02

(52) U.S. Cl. CPC A61K 8/602; A61K 8/37; A61K 8/604; A61Q 15/00; A61Q 17/02; A61Q 17/04; A61Q 19/007; A61Q 19/08; A61K 2800/30

(57) ABSTRACT

A composition contains 2 to 6 parts by weight of at least one rhamnolipid, 1 to 4 parts by weight of at least one alkyl polyglycoside, and 1 to 3 parts by weight of at least one acyl lactylate.

### FIELD OF THE INVENTION

The invention relates to compositions comprising
A) 2 to 6 parts by weight of at least one rhamnolipid,
B) 1 to 4 parts by weight of at least one alkyl polyglycoside, and
C) 1 to 3 parts by weight of at least one acyl lactylate.

### PRIOR ART

One possibility of being able to solubilize hydrophobic substances in aqueous formulations is to use hydrotropic substances. Non-ionic and ionic surfactants are suitable for this purpose. Solubilizers used today include ethoxylated fatty esters such as PEG-40 hydrogenated castor oil and polysorbate 20. These have a wide range of applications and can be effective over a wide pH range. PEG-containing substances like the above are based on petrochemical raw materials. For some years, PEG-containing substances have increasingly been avoided in cosmetic formulations. Ethoxylated triglycerides such as PEG-40 hydrogenated castor oil have a high viscosity at room temperature which in turn has an influence on the processability.

In the course of using sustainable raw materials, alternative solubilizers or solubilizer preparations have been developed in recent years which dispense with the use of ethoxylated ingredients. These include polymers such as polyglycerol partial esters as disclosed, for example, in WO2012007754 and EP0835862. These substances usually have a high viscosity and accompanying poor manageability. Furthermore, the effectiveness is usually very selective. JP2001232174 discloses the combination of polyglycerol esters and salts of esters of lactic acid and fatty acids.

Alkyl polyglycosides are suitable solubilizers, likewise acylated amino acids that are described, for example, in EP2276453B1.

Alkyl polyglucosides and acylated amino acids, such as acyl proline, only exhibit good solubilizing performance in a certain pH range and show high selectivity with respect to the solute, especially for essential oils.

WO2010106423 discloses solubilizing compositions comprising a combination of a) polyglycerol esters of fatty acids, wherein the degree of polymerization of the polyglycerol is between 2.5 and 5.5 and the number of carbon atoms in the fatty acid is between 8 and 10; and b) acyl glutamates having an acid radical consisting of 8 to 14 carbon atoms, which are present with alkali metal, alkaline earth metal or ammonium ions or with amines; in ratios by weight between components a) and b) between 5:95 and 95:5. The field of application remains limited to essential oils.

EP2366376 describes improved solubilizing performance using wetting agents in combination with alkyl polyglucosides. In the latter two examples mentioned, particularly good solubilizing performances are only exhibited using complex mixtures of surfactants. The mixtures also exhibit strong foaming behaviour.

To produce all of these substances, the use of tropical oils is required. Some of these substances are not readily biodegradable.

EP2786742 discloses cosmetic formulations comprising at least one rhamnolipid.

The object of the invention was to provide solubilizers which are able to overcome at least one disadvantage of the solubilizers of the prior art.

### DESCRIPTION OF THE INVENTION

It has been found, surprisingly, that a composition of rhamnolipids, alkyl polyglycosides and lactic acid derivatives exhibit excellent solubilizing performance and that are able to address the object of the invention.

The composition according to the invention has the advantage that it can be formulated free of PEG.

It is furthermore advantageous that this can be produced exclusively from sustainable raw materials.

A further advantage of the present invention is that the compositions according to the invention are not based on petrochemical raw materials.

An advantage compared to solubilizers such as PEG-40 hydrogenated castor oil is the comparatively low viscosity and the accompanying improved handling of the composition according to the invention.

An advantage of the present invention is that it can already produce clear solutions of essential oils at lower use concentrations.

Furthermore, the compositions according to the invention have proven to be exceptionally stable over a long period.

A further advantage of the compositions according to the invention is the inherent preservation, which renders the use of preservatives superfluous, as used in some of the individual components. The compositions according to the invention further exhibit advantageous foaming behavior compared to the individual raw materials or other solubilizers.

The compositions according to the invention are characterized by improved skin feel.

In the case of make-up removal, the compositions according to the invention exhibit advantages compared to alternative solubilizers.

Compared to the mild raw materials of the compositions according to the invention and in comparison to other solubilizers, the compositions according to the invention are characterized by their mildness, such as reduced protein denaturation in the Zein test or RBC test.

The present invention therefore relates to compositions comprising
A) 2 to 6 parts by weight of at least one rhamnolipid,
B) 1 to 4, preferably 1 to 3, parts by weight of at least one alkyl polyglycoside, and
C) 1 to 3 parts by weight of at least one acyl lactylate.

The invention further relates to the use of the composition according to the invention for solubilizing a cosmetic oil.

The compositions according to the invention will be described by way of example below, without any intention that the invention be restricted to these illustrative embodiments. Where ranges, general formulae or classes of compounds are specified below, these are intended to encompass not only the corresponding ranges or groups of compounds which are explicitly mentioned but also all subranges and subgroups of compounds which can be obtained by removing individual values (ranges) or compounds. Where documents are cited within the context of the present description, the entire content thereof is intended to be part of the disclosure-content of the present invention. Where, in the context of the present invention, compounds, for instance organomodified polysiloxanes, are described which can have different units multiple times, then these can occur in random distribution (random oligomer) or ordered (block oligomer) in these compounds. The information regarding the number of units in such compounds is to be understood as an average averaged over all corresponding compounds.

The term “rhamnolipid” in the context of the present invention encompasses rhamnolipids, protonated forms thereof and also in particular salts thereof.

The term “rhamnolipid” in the context of the present invention is understood to mean particularly mixtures of compounds of the general formula (I) and salts thereof,

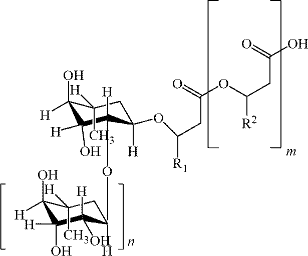

where
m = 2, 1 or 0,
n = 1 or 0,
R¹ and R² = mutually independently identical or different organic radical having 2 to 24, preferably 5 to 13 carbon atoms, in particular optionally branched, optionally substituted, particularly hydroxy-substituted, optionally unsaturated, in particular optionally mono-, bi- or tri-unsaturated alkyl radical, preferably selected from the group consisting of pentenyl, heptenyl, nonenyl, undecenyl and tridecenyl and (CH₂)ₒ—CH₃ where o = 1 to 23, preferably 4 to 12.

If n = 1, the glycosidic bond between the two rhamnose units is preferably in the a-configuration. The optically active carbon atoms of the fatty acids are preferably present as R-enantiomers (e.g. (R)-3-{(R)-3-[2-0-(a-L-rₕamnopyranosyl)-a-L-rhamnopyranosyl]oxydecanoyl}oxydecanoate).

The term “di-rharnriolipid” in the context of the present invention is understood to mean compounds of the general formula (I) or salts thereof, where n = 1 .

The term “mono-rhamnolipid” in the context of the present invention is understood to mean compounds of the general formula (I) or salts thereof, where n = 0.

Distinct rhamnolipids are abbreviated according to the following nomenclature:
“diRL-CXCY” is understood to mean di-rhaninolipids of the general formula (I), in which one of the radicals R¹ and R² = (CH₂)ₒ—CH₃ where o = X-4 and the remaining radical R¹ or R² = (CH₂)ₒ—CH₃ where o = Y-4.
“monoRL-CXCY” is understood to mean mono-rhamnolipids of the general formula (I), in which one of the radicals R¹ and R² = (CH₂)ₒ—CH₃ where o = X-4 and the remaining radical R¹ or R² = (CH₂)ₒ—CH₃ where o = Y-4.

The nomenclature used therefore does not distinguish between “CXCY” and “CYCX”.

For rhamnolipids where m=0, monoRL-CX or diRL-CX is used accordingly.

If one of the aforementioned indices X and/or Y is provided with “:Z”, this signifies that the respective radical R¹ and/or R² is equal to an unbranched, unsubstituted hydrocarbon radical having X-3 or Y-3 carbon atoms having Z double bonds.

To determine the content of rhamnolipids in the context of the present invention, only the mass of the rhamnolipid anion is considered, i.e. “general formula (I) less one hydrogen”.

To determine the content of rhamnolipids in the context of the present invention, all rhamnolipids are converted by acidification into the protonated form (cf. general formula (I)) and quantified by HPLC.

The term “alkyl polyglycoside” in the context of the present invention is understood to mean reaction products of sugars and/or polymers thereof, such as starch, for example, with fatty alcohol. The term “acyl lactylate” in the context of the present invention is understood to mean salt-form reaction products of fatty acid with lactic acid and/or polylactic acid.

Unless stated otherwise, all percentages (%) given are percentages by mass.

It is preferred according to the invention that the compositions comprise 51% by weight to 95% by weight, preferably 70% by weight to 90% by weight, particularly preferably 75% by weight to 85% by weight, of diRL-C10C10, where the percentages by weight refer to the sum total of all rhamnolipids present.

It is preferred according to the invention that the compositions comprise 0.5%., by weight to 9% by weight, preferably 0.5% by weight to 3% by weight, particularly preferably 0.5% by weight to 2% by weight, of monoRL-C10C10, where the percentages by weight refer to the sum total of all i*hainiiolipids present.

Preferred compositions according to the invention are characterized in that the ratio by weight of all di-rhamnolipids present to all mono-rhamnolipids present is greater than 51:49, particularly greater than 91:9, preferably greater than 97:3, particularly preferably greater than 98:2.

It is preferred according to the invention that the compositions comprise 0.5 to 25% by weight, preferably 5% by weight to 15% by weight, particularly preferably 7% by weight to 12% by weight, of diRL-C1OC12, where the percentages by weight refer to the sum total of all rharnriolipids present.

It is preferred according to the invention that the compositions comprise 0.1% by weight to 5% by weight, preferably 0.5% by weight to 3% by weight, particularly preferably 0.5% by weight to 2% by weight, of monoRL-C10C12 and/or, preferably and, 0.1 % by weight to 5% by weight, preferably 0.5% by weight to 3% by weight, particularly preferably 0.5% by weight to 2% by weight, of monoRL-C10C12:1, where the percentages by weight refer to the sum total of all rhamnolipids present.

Particularly preferred compositions according to the invention are characterized in that they comprise
0.5% by weight to 15% by weight, preferably 3% by weight to 12% by weight, particularly preferably 5% by weight to 10% by weight, of diRL-C10C12:1,
0.5 to 25% by weight, preferably 5% by weight to 15% by weight, particularly preferably 7% by weight to 12% by weight, of diRL-C10C12,
0.1% by weight to 5% by weight, preferably 0.5% by weight to 3% by weight, particularly preferably 0.5% by weight to 2% by weight, of monoRL-C10C12 and
0.1% by weight to 5% by weight, preferably 0.5% by weight to 3% by weight, particularly preferably 0.5% by weight to 2% by weight, of monoRL-C10C12:1,
where the percentages by weight refer to the sum total of all rhamnolipids present.

It is moreover preferred if the composition according to the invention comprises only small amounts of rhamnolipids of the formula monoRL-CX or diRL-CX. In particular, the composition according to the invention preferably comprises

0% by weight to 5% by weight, preferably 0% by weight to 3% by weight, particularly preferably 0.1 % by weight to 1% by weight, of diRLC1 0, where the percentages by weight refer to the sum total of all rhamnolipids present, and the term “0% by weight” is understood to mean no detectable amount.

Preferred compositions according to the invention are characterized in that component B) is selected from alkyl polyglycosides having an alkyl chain comprising 4 to 22 carbon atoms, preferably 6 to 14 carbon atoms, particularly preferably 6 to 10 carbon atoms.

Preferred compositions according to the invention are characterized in that component B) is selected from alkyl polyglycosides having a polyglycoside portion having 1 to 12 glucose units, preferably 1.2 to 8 glucose units, particularly preferably 1.4 to 1.8 glucose units.

Preferred compositions according to the invention are characterized in that component B) is selected from at least one of the group comprising, preferably consisting of, caprylylicapryl glucoside, coco glucoside, lauryl glucoside and decyl glucoside.

The compositions according to the invention comprise component C) acyl lactylate.

These are salts and therefore comprise cations.

Preferred compositions according to the invention are characterized in that component C) is selected from at least one of acyl lactylates comprising at least one cation, selected from the group comprising, preferably consisting of, Li⁺, K⁺, Mg²⁺, Ca²⁺, Al³⁺, NH₄⁺, primary ammonium ions, secondary ammonium ions, tertiary ammonium ions and quaternary ammonium ions.

Exemplary representatives of suitable ammonium ions are tetramethylammonium, tetraethylammonium, tetrapropylammonium, tetrabutylammonium and [(2-hydroxyethyl)trimethylammonium] (choline) and also the cations of 2-aminoethanol (ethanolamine, MEA), diethanolamine (DEA), 2,2′,2″-nitrilotriethanol (triethanolamine, TEA), 1-aminopropan-2-ol (monoisopropanolamine), ethylenediamine, diethylenetriamine, triethylenetetramine, tetraethylenepentamine, 1,4-diethylenediamine (piperazine), aminoethylpiperazine and aminoethylethanolamine.

Particularly preferred cations are selected from the group comprising, preferably consisting of, Li⁺, Al³⁺, NH₄⁺ and the triethanolammonium cation.

Preferred compositions according to the invention are characterized in that component C) is selected from at least one of acyl lactylates having an acyl chain of a natural fatty acid, particularly comprising 4 to 22 carbon atoms, preferably 6 to 16 carbon atoms, particularly preferably 8 to 14 carbon atoms.

Preferred compositions according to the invention are characterized in that component C) is selected from at least one of the group comprising, preferably consisting of, sodium caproyl lactylate, sodium lauroyl lactylate and sodium stearoyl lactylate, sodium oleoyl lactylate, sodium behenoyl lactylate, sodium isostearoyl lactylate and sodium cocoyl lactylate.

Preferred compositions according to the invention are characterized in that said compositions have a pH at 25° C. of 3.5 to 8.5, preferably 4.5 to 7.4, particularly preferably 5.0 to 6.0.

The “pH” in connection with the present invention is defined as the value which is measured for the relevant composition at 25° C. after stirring for five minutes using a pH electrode calibrated in accordance with ISO 4319 (1977).

Preferred compositions according to the invention are characterized in that the sum total of components A), B) and C), based on the total composition, make up at least 90% by weight, preferably at least 95% by weight, particularly preferably at least 98% by weight.

These preferred compositions according to the invention are concentrates, which may be used advantageously for producing formulations, especially cosmetic formulations.

In an alternative preferred embodiment of the compositions according to the invention, these take the form of ready-to-use formulations; these preferred alternative compositions according to the invention are characterized in that the components A), B) and C) are present in sum total of 0.1% by weight to 40% by weight, preferably 0.3% by weight to 35% by weight, particularly preferably 0.5% by weight to 10% by weight, wherein the percentages by weight refer to the total composition.

These preferred alternative compositions according to the invention are ready-to-use formulations, especially cosmetic formulations, which may result in advantageous preparation with concentrates specified above.

Therefore, the present invention further relates to the use of the compositions according to the invention for producing formulations, especially cosmetic formulations.

Preferred ready-to-use formulations according to the invention comprise, in addition to the compositions according to the invention, at least one further surfactant, in which case, for example, anionic, nonionic, cationic and/or amphoteric surfactants can be used. From an applications point of view, preference is given to mixtures of anionic and nonionic surfactants. The total surfactant content of the aqueous formulation is preferably 5 to 60% by weight and particularly preferably 15 to 40% by weight, based on the total formulation.

The ready-to-use formulations according to the invention can further comprise at least one additional component selected from the group of
emollients,
emulsifiers,
thickeners/viscosity regulators/stabilizers,
UV light protection filters,
antioxidants,
hydrotropes (or polyols),
solids and fillers,
film formers,
pearlescence additives,
deodorant and antiperspirant active ingredients,
insect repellents,
self-tanning agents,
preservatives,
conditioning agents,
perfumes,
dyes,
odor absorbers,
cosmetic active ingredients,
care additives,
superfatting agents,
solvents.

Substances which can be used as exemplary representatives of the individual groups are known to those skilled in the art and can be found for example in German application DE 102008001788.4. This patent application is hereby incorporated as reference and thus forms part of the disclosure.

As regards further optional components and the amounts used of these components, reference is made expressly to the relevant handbooks known to those skilled in the art, for example K. Schrader, “Grundlagen und Rezepturen der Kosmetika [Cosmetics - fundamentals and formulations]”, 2nd edition, pages 329 to 341, Hüthig Buch Verlag Heidelberg.

The amounts of the particular additives are determined by the intended use.

Typical boundary formulations for the respective applications are known prior art and are contained for example in the brochures of the manufacturers of the particular base and active ingredients. These existing formulations can generally be adopted unchanged. However, if required, for adjustment and optimization, the desired modifications can be undertaken by simple tests without complication.

Preferred ready-to-use formulations according to the invention are characterized in that said formulations comprise

D) 0.01% by weight to 40% by weight, preferably 0.1% by weight to 30% by weight, particularly preferably 0.2% by weight to 2% by weight, of at least one cosmetic oil,

where the percentages by weight refer to the total composition.

Cosmetic oils that can be used are substances such as silicone oils, functionalized silicones, mineral oils, fatty acid esters, natural oils such as vegetable oils, animal oils, fragrance oils, essential oils and aromas, and mixtures thereof.

In this context, the cosmetic oil is preferably selected from at least one of the group comprising, preferably consisting of, cyclopentasiloxane, cyclomethicone, dimethicone, dimethiconol, amodimethicone, PEG/PPG dimethicones, cetyl dimethicone, stearyl dimethicone, stearoxy dimethicone, behenoxy dimethicone, polyisobutene, petrolatum, mineral oil, hydrogenated polydodecene, hydrogenated polydecene, polydecene, isoamyl cocoate, PPG-3 myristyl ether, PPG-11 stearyl ether, dicaprylyl ether, dicaprylyl carbonate, cetearyl isononanoate, cetyl ethylhexanoate, diethyhexyl carbonate, cetyl ricinoleate, myristyl myristate, stearyl heptanoate, decyl cocoate, decyl oleate, PPG-15 stearyl ether, octyldodecanol, isocetyl palmitate, cetearyl ethylhexanoate, ethylhexyl palmitate, ethylhexyl stearate, isopropyl palmitate, PPG-14 butyl ether, triisostearin, C12-15 alkyl benzoate, phenoxyethyl caprylate, isopropyl myristate, caprylic/capric triglyceride, sunflower oil, olive oil, argan oil, mineral oil, castor oil, ricinus oil, cocoa oil, palm oil, coconut oil, avocado oil, almond oil, jojoba oil, corn oil, rapeseed oil, sesame oil, soya oil, wheatgerm oil, walnut oil, oleyl erucate, cineol, thymol, menthol, methyl salicylate, wintergreen oil, carvacrol, camphor, anethole, carvone, eugenol, isoeugenol, limonene, ocimene, n-decyl alcohol, methyl acetate, citronellyl acetate, methyl eugenol, linalool, ethyl linalool, Safrola vanillin, mint oil, peppermint oil, lemon oil, orange oil, sage oil, rosemary oil, cinnamon oil, pimento oil, laurel oil, cedar oil, gerianol, verbenone, anise oil, benzaldehyde, dergamot oil, bitter almond, chlorothymol, cinnamaldehyde, lemongrass oil, clove oil, eucalyptus oil, guaiacol, lavender oil, mustard oil, phenol, phenyl salicylate, pine oil, spruce needle oil, sassafras oil, spike lavender oil, styrax, thyme oil, turpentine oil, Star anise oil, almond oil, caraway oil, cardamom oil, celery oil, camomile oil, coriander oil, cottonseed oil, cumin oil, dill oil, fennel oil, garlic oil, geranium oil, ginger oil, grapefruit oil, lime oil, linseed oil, mint oil, parsley oil, pepper oil, rose oil, soybean oil and turmeric oil.

Preferred ready-to-use formulations according to the invention are characterized in that said formulations have a pH at 25° C. of 3.5 to 9, preferably 4 to 7, particularly preferably 4.5 to 6.5.

Preferred ready-to-use formulations according to the invention are those characterized in that the formulation is essentially free of polyethers and compounds comprising polyethers.

In the context of the present invention, the term “essentially free of polyethers and compounds comprising polyethers” describes the fact that compounds present only in traces, preferably do not comprise any alkoxy groups, oligoalkoxy groups or polyalkoxy groups such ethylene oxide or propylene oxide for example. The concentration of compounds comprising polyethers should be less than 0.1% by weight, particularly preferably less than 0.01% by weight, based on the total formulation, preferably below the detection limit of customary analytical methods such as NMR spectroscopy, GPC or MALDI.

The present invention further provides a process for solubilizing a cosmetic oil comprising the process steps of
A) providing a composition according to at least one of Claims 1 to 6,
B) bringing the composition into contact with at least one cosmetic oil
C) mixing the composition with the cosmetic oil.

It is preferable in accordance with the invention if process step C) is conducted in a temperature range of 15° C. to 40° C., preferably of 20° C. to 30° C., particularly preferably of 22° C. to 27° C.

The present invention further relates to the use of a composition according to the invention for solubilizing a cosmetic oil.

The compositions according to the invention and the formulations according to the invention comprising the compositions according to the invention can advantageously be used for cleaning surfaces, e.g. for cleaning leather. For this form of the use according to the invention, the surface is preferably the surface of a living being, in particular a person, with such surfaces being particularly preferably selected from skin and hair, and in which the use is non-therapeutic.

For the uses according to the invention and the processes according to the invention, preference shall be given to compositions according to the invention corresponding to the above preferred embodiments of the compositions according to the invention.

The same applies to the cosmetic oils.

The examples adduced hereinafter illustrate the present invention by way of example, without any intention of restricting the invention, the scope of application of which is apparent from the entirety of the description and the claims, to the embodiments specified in the examples.

### EXAMPLES

### Example 0a: Production of Rhamnolipid a

A fermentation was carried out using a Pseudomonas putida strain pBBR1MCS2-Plac-rh/ABC-T-Ptac-rh/C-T comprising the rhamnolipid biosynthesis genes RhIA, RhIB and RhIC, the preparation of which is described in US2014295168. The pre-culture was carried out in shaking flasks as described in WO2012013554A1. A mineral medium (M9) was also used for the main culture. The fermentation was conducted in a 2 litre fermenter in a carbon-limited manner via a glucose supplementary feed. The glucose supplementary feed was conducted on the basis of the dissolved oxygen signal. The dissolved oxygen was regulated at 20% saturation via the stirrer speed. The pH was regulated at 7 via a pH electrode and addition of 2 M sulfuric acid or a 20% by weight ammonia solution. In order to prevent the fermentation broth foaming over, the defoamer DOW Corning 1500 was metered in as required. The fermentation was conducted over 4 days up to a dry biomass of 15 g/l. The rhamnolipid concentration was determined by HPLC and was 9.8 g/l. After separating off the cells by centrifugation at 10.000 g, the fermentation broth was adjusted to a pH of 3.1 by addition of concentrated H₂SO₄. By centrifugation again at 500 g, a pasty solid concentrate was obtained having an RL proportion of 45% by weight and with a viscosity of > 10 000 mPas. With continuous stirring, a 50% by weight aqueous KOH solution was added to the pasty suspension of the concentrated rhamnolipid precipitate and the pH was adjusted to 6. Here, the pasty mass liquified, which was accompanied by a sharp drop in viscosity.

Rhamnolipid species detected by HPLC were: RL total [%] (HPLC) 91 diRL-C8C10 13.9 monoRL-C8C10 0.51 diRL-C10C10 61.4 monoRL-C10C10 1.4 diRL-C10C12:1 5.9 diRL-C10C12 5.5 otherRL 2.2

### Example 0b: Production of Rhamnolipid b

The 50% by weight rhamnolipid solution produced as described in Example 0a was diluted to 1% by addition of water. Two litres of this solution were heated to 50° C. With gentle stirring, 200 units of a thermally stable rhamnosidase (ThermoActiveTM Rhamnosidase A, Prokazyme) were added and the reaction was carried out overnight. After 20 h, a sample of the solution was analysed by HPLC. The di-rhamnolipid had been completely converted to mono-rhamnolipid and rhamnose. Subsequently, the enzyme was inactivated for one hour at 80° C. The whole batch was then freeze-dried. The freeze-dried product was adjusted to a mono-rhamnolipid active content of 50% by weight by addition of water.

### Example 0c: Production of Rhamnolipid c

A mixture of 33% by weight of the composition of example 0b was stirred with 67% of the composition of example 0a at room temperature for 1 h.

### Example 1

The dissolution capacity of the solubilizer mixture was investigated by mixing this with cosmetic oils and then diluting with water. The minimal amount of solubilizer was found which was necessary to clearly dissolve 1 % of the specified oil in water. For this purpose, a test series of a particular amount of the solubilizer mixture (1 g–21 g) was mixed with 1 g of the relevant oil. This was then made up to 100 g with water. The sample was assessed visually. In addition, the turbidity values were determined by turbidity measurements (HACH 2100AN IS Turbidimeter) in 11 mm glass cuvettes. All samples which revealed a turbidity value of <30 NTU were assessed as clear. After cooling to 20° C., a “clear mixture” must not become turbid again over a period of 2 days.

In Table 1, the resulting mass ratios of solubilizer to oil which were required to obtain clear mixtures are summarized.

| | | | |
|---|---|---|---|
| Active content, to 100% H₂O | Lemon oil | Tegosoft M | Lemongrass oil |
| Caprylyl/Capryl Glucoside (55.8%) | 7% | 22% | 10% |
| Sodium Caproyl/Lauroyl Lactylate (10%)¹ | | | |
| Rhamnolipid a (16.25%) | 5% | 20% | 10% |
| Caprylyl/Capryl Glucoside (35.65%) | | | |
| Sodium Caproyl/Lauroyl Lactylate (10%) ¹ | | | |
| Rhamnolipid a (22.5%) | 4% | 21% | 9% |
| Caprylyl/Capryl Glucoside (27.9%) | | | |
| Sodium Caproyl/Lauroyl Lactylate (10%) ² | | | |
| Rhamnolipid a (25%) Caprylyl/Capryl | 6% | 14% | 7% |
| Glucoside (21.7%) | | | |
| Sodium Caproyl/Lauroyl Lactylate (15%) ² | | | |
| Rhamnolipid a (28.75%) | 5% | 16% | 8% |
| Coco Glucoside (20.15%) | | | |
| Sodium Caproyl/Lauroyl Lactylate (10%) ² | | | |
| Rhamnolipid c (25%) | 5% | 15% | 8% |
| Caprylyl/Capryl Glucoside (21.7%) | | | |
| Sodium Caproyl/Lauroyl Lactylate (15%) ² | | | |

Rhamnolipid a: described in Example 0a
Rhamnolipid c: described in Example 0c
Caprylyl/Capryl Glucoside PLANTACARE® 810 UP BASF Personal Care and Nutrition GmbH, 62% solution
Coco Glucoside PLANTACARE® 818 BASF Personal Care and Nutrition GmbH, 52% solution
Sodium Caproyl/Lauroyl Lactylate dermosoft® decalact Evonik Dr. Straetmans GmbH
Lemon oil Frey + Lau GmbH
Isopropyl Myristate TEGOSOFT® M Evonik Nutrition & Care GmbH
Lemongrass oil Carl Roth GmbH + Co. KG
¹ noninventive examples
² inventive examples

1. A composition, comprising the following components:
A) 2 to 6 parts by weight of at least one rhamnolipid,
B) 1 to 4 parts by weight of at least one alkyl polyglycoside, and
C) 1 to 3 parts by weight of at least one acyl lactylate.

2. The composition according to claim 1, wherein component B) comprises at least one alkyl polyglycoside having an alkyl chain comprising 4 to 22 carbon atoms.

3. The composition according to claim 1, wherein component B) comprises at least one alkyl polycoside with a polyglycoside portion having 1 to 12 glucose units.

4. The composition according to claim 1, wherein component B) is selected from at least one of the group consisting of caprylyl/capryl glucoside, coco glucoside, lauryl glucoside, and decyl glucoside.

5. The composition according to claim 1, wherein component C) comprises at least one acyl lactylate having an acyl chain of a natural fatty acid.

6. The composition according to claim 1, wherein component C) is selected from at least one of the group consisting of sodium caproyl lactylate, sodium lauroyl lactylate, sodium stearoyl lactylate, sodium oleoyl lactylate, sodium behenoyl lactylate, sodium isostearoyl lactylate, and sodium cocoyl lactylate.

7. The composition according to claim 1, wherein a sum total of components A), B), and C), based on a total composition, make up at least 90% by weight.

8. The composition according to claim 1, wherein components A), B), and C) are present in a sum total of 0.1% by weight to 40% by weight, wherein percentages by weight refer to a total composition.

9. The composition according to claim 8, wherein said composition further comprises the following component:
D) 0.01% by weight to 40% by weight of at least one cosmetic oil, wherein the percentages by weight refer to the total composition.

10. The composition according to claim 2, wherein the at least one cosmetic oil is selected from the group consisting of cyclopentasiloxane, cyclomethicone, dimethicone, dimethiconol, amodimethicone, a PEG/PPG dimethicone, cetyl dimethicone, stearyl dimethicone, stearoxy dimethicone, behenoxy dimethicone, polyisobutene, petrolatum, mineral oil, hydrogenated polydodecene, hydrogenated polydecene, polydecene, isoamyl cocoate, PPG-3 myristyl ether, PPG-11 stearyl ether, dicaprylyl ether, dicaprylyl carbonate, cetearyl isononanoate, cetyl ethylhexanoate, diethyhexyl carbonate, cetyl ricinoleate, myristyl myristate, stearyl heptanoate, decyl cocoate, decyl oleate, PPG-15 stearyl ether, octyldodecanol, isocetyl palmitate, cetearyl ethylhexanoate, ethylhexyl palmitate, ethylhexyl stearate, isopropyl palmitate, PPG-14 butyl ether, triisostearin, C12-15 alkyl benzoate, phenoxyethyl caprylate, isopropyl myristate, caprylic/capric triglyceride, sunflower oil, olive oil, argan oil, mineral oil, castor oil, ricinus oil, cocoa oil, palm oil, coconut oil, avocado oil, almond oil, jojoba oil, corn oil, rapeseed oil, sesame oil, soya oil, wheatgerm oil, walnut oil, oleyl erucate, cineol, thymol, menthol, methyl salicylate, wintergreen oil, carvacrol, camphor, anethole, carvone, eugenol, isoeugenol, limonene, ocimene, n-decyl alcohol, methyl acetate, citronellyl acetate, methyl eugenol, linalool, ethyl linalool, Safrola vanillin, mint oil, peppermint oil, lemon oil, orange oil, sage oil, rosemary oil, cinnamon oil, pimento oil, laurel oil, cedar oil, gerianol, verbenone, anise oil, benzaldehyde, dergamot oil, bitter almond, chlorothymol, cinnamaldehyde, lemongrass oil, clove oil, eucalyptus oil, guaiacol, lavender oil, mustard oil, phenol, phenyl salicylate, pine oil, spruce needle oil, sassafras oil, spike lavender oil, styrax, thyme oil, turpentine oil, Star anise oil, almond oil, caraway oil, cardamom oil, celery oil, camomile oil, coriander oil, cottonseed oil, cumin oil, dill oil, fennel oil, garlic oil, geranium oil, ginger oil, grapefruit oil, lime oil, linseed oil, mint oil, parsley oil, pepper oil, rose oil, soybean oil, and turmeric oil.

11. The composition according to claim 8, wherein said composition has a pH at 25° C. of 3.5 to 8.5.

12. The composition according to claim 8, wherein the composition is essentially free of poly ethers and compounds comprising polyethers.

13. A process for solubilizing a cosmetic oil, comprising:
providing the composition according to claim 1,
bringing the composition into contact with at least one cosmetic oil, and
mixing the composition with the at least one cosmetic oil.

14. A cosmetic formulation, comprising:
1,the composition according to claim 1,
a cosmetic oil, and
at least one additional component.

15. A method, comprising:
cleaning a surface with the composition according to claim 1 .

16. The composition according to claim 1, wherein the composition comprises 1 to 3 parts by weight of component B).

17. The composition according to claim 2, wherein the at least one alkyl polyglycoside has an alkyl chain comprising 6 to 10 carbon atoms.

18. The composition according to claim 5, wherein the at least one acyl lactylate has an acyl chain of natural fatty acid comprising 4 to 22 carbon atoms.

19. The composition according to claim 8, wherein the composition comprises 0.5% by weight to 10% by weight of components A), B), and C).

20. The composition according to claim 9, wherein the composition comprises 0.2% by weight to 2% by weight of component D).
